(19) United States
(12) Patent Application Publication
(10) Pub. No.: US 2022/0378957 A1
(43) Pub. Date: Dec. 1, 2022

(54) Storage Compartment

(71) Applicant: Polymer Optics Automotive Limited, Wolston (GB)

(72) Inventors: Michael John Hanney, Calne (GB); Andrew Jonathan Dennington, Horsell (GB); Matthew Powell, Wombourne (GB); Ian Michael Bryant, Worle (GB)

(21) Appl. No.: 17/329,964

(22) Filed: May 25, 2021

(51) Int. Cl. A61L 2/10; B60R 7/04

(52) U.S. Cl. CPC A61L 2/10; B60R 7/04; A61L 2202/122; A61L 2202/16; A61L 2202/14

(57) ABSTRACT

A storage compartment for a vehicle includes a body defining a sterilization volume configured to receive an item to be sterilized. The body includes a lower wall, and a side wall extending from the lower wall. A lid is coupled with the at least one of the walls and is configured to selectively close the sterilization volume. A lighting assembly is provided, to direct emitted light into the sterilization volume.

### FIELD OF THE DISCLOSURE

The present disclosure generally relates to a storage compartment for a vehicle, and more specifically to a storage compartment for use within a vehicle center console.

### BACKGROUND OF THE DISCLOSURE

Vehicle consoles typically include a center stack and a center console. The center stack and the center console include various storage compartments.

### SUMMARY OF THE DISCLOSURE

According to one aspect of the present disclosure, a storage compartment for a vehicle includes a body defining a sterilization volume configured to receive an item to be sterilized. The body includes a lower wall, and a side wall extending from the lower wall. A lid is coupled with the at least one of the walls and is configured to selectively close the sterilization volume. A lighting assembly is provided, comprising a plurality of light sources in the lid and a plurality of light sources in the lower wall, the light sources arranged to direct emitted light into the sterilization volume.

Embodiments of this aspect of the disclosure can include any one or a combination of the following features:
a hinge to hingedly couple the lid to the side wall;
the lid is arranged to seal the sterilization volume when closed;
the lid is coupled to the side wall, such as an upper portion of the side wall;
the lid is configured to selectively close an opening at the top of the sterilization volume;
the plurality of light sources in the lid comprises more than 2 light sources, for example more than 5, such as more than 9;
the plurality of light sources in the lid comprises 10 light sources
the plurality of light sources in the lower wall comprises more than 2 light sources, for example more than 5, such as more than 9;
the plurality of light sources in the lower wall comprises 10 light sources;
light source(s) in the lid comprising UV-C emitters;
light source(s) in the lower wall comprising UV-C emitters;
light source(s) in the lid comprising low-power emitters;
light source(s) in the lower wall comprising low-power emitters;
light source(s) in the lid to emit at 200-280 nm wavelength;
light source(s) in the lid to emit with a Lambertian output profile;
light source(s) in the lower wall to emit 2.5 mW each;
light source(s) in the lower wall to emit at 200-280 nm wavelength;
light source(s) in the lower wall to emit with a Lambertian output profile;
light source(s) in the lower wall to emit 2.5 mW each;
light sources(s) collectively to emit less than 75 mW of UV-C radiation;
light sources(s) collectively to emit greater than 25 mW of UV-C radiation;
light sources(s) collectively to emit 50 mW of UV-C radiation;
the light sources in the lid being arranged in a spaced array, for example an array of three rows;
the light sources in the lower being arranged in a spaced array, for example an array of three rows;
a support coupled within the sterilization volume;
the support removably coupled within the sterilization volume;
the support comprising a plurality of openings therethrough to allow light from the light sources to pass therethrough;
the openings having a characteristic dimension of between 5 mm and 10 mm, such as 8 mm, the characteristic dimension being for example the length of one or more sides of the openings;
the openings being generally rectangular when viewed in the plane of the support, for example generally square;
the openings defined by a non-planar perimeter;
the support formed from a mesh material;
the support comprising a wire mesh;
the support comprising a mesh formed of 1 mm diameter wire;
the support comprising a mesh of metallic material;
the support comprising a stainless steel;
the support comprising a 304 grade stainless steel;
the support formed as a basket;
the support comprising an electro-chemically polished finish; —the support defining a support portion for the item to be sterilized, such as on which the item to be sterilized rests, in use;
the support portion being spaced from the lower wall and spaced from the lid when the sterilization volume is closed, such as substantially midway between lower wall and the lid.
the support portion being substantially parallel to the lower wall;
the support portion being spaced from the side wall;
the support portion being substantially central to the sterilization volume;
the support comprising a coupling portion extending from support portion, thereby the support is coupled to side wall;
the coupling portion extending from the support portion at an angle to the support portion, for example substantially perpendicular to the support portion;
the coupling portion extending from more than one side of the support portion;
the coupling portion extending to couple with the side wall, such as proximate to the lid
the coupling portion extending to couple to the side wall at the top of the side wall;
the support comprising an optically matt finish in the visible spectral range;
the support portion comprising an out of plane displacement, such as a displacement above, or a displacement below the support plane;

the out of plane displacement resulting in a non-planar perimeter for openings in the support portion;
the support portion comprising a plurality of out of plane displacements;
the support portion comprising a plurality of out of plane displacements;
the support portion comprising an out of plane displacement projecting toward the lower wall;
the support portion comprising an out of plane displacement projecting toward the lower wall;
the support portion comprising a curved surface, such as without discontinuity of curvature;
the support portion comprising a ripple pattern, with an out of plane displacement in the form of a ripple projecting towards the lower wall, and/or an out of plane displacement in the form of a ripple projecting toward the lid;
the support portion comprising a ripple pattern, with ripples projecting towards the lower wall, and/or ripples projecting toward the lid;
the support portion comprising ripple pattern, with a first arrangement of ripples projecting toward the lower wall and/or a second arrangement of ripples projecting toward the lid;
the support portion comprising a ripple pattern, with ripples of the first arrangement of ripples arranged between ripples of the second arrangement of ripples;
the support portion comprising a two-dimensional ripple pattern, with ripples of the first arrangement of ripples arranged between ripples of the second arrangement of ripples considering a first direction, and considering a second direction non-parallel to the first direction;
the support portion comprising a two-dimensional ripple pattern, with ripples of the first arrangement of ripples arranged between ripples of the second arrangement of ripples considering a first direction, and considering a second direction perpendicular to the first direction;
the light sources in the lid being arranged with at least one light source located directly above the support;
the light sources in the lid being arranged with at least one light source located directly above the coupling portion;
the light sources in the lid being arranged with at least one light source located directly above the support;
the light sources in the lid being arranged with at least one light source located directly above the coupling portion;
the light sources in the lower wall being arranged with at least one light source located directly below the support portion;
the light sources in the lower wall being arranged with at least one light source located directly below the coupling portion;
an inward facing surface of one or more of the lower wall, side wall and lid comprising an optically matt finish in the visible spectral range;
the inward facing surface of one or more of the lower wall, side wall and lid comprises a reflective finish in the UV-C range;
an inward facing surface of one or more of the lower wall, side wall and lid comprising a metallic material, such as an aluminum material with an anodized coating and/or a physical vapor deposition finish applied thereto in order to provide an optically matt finish in the visible range, but a finish that is reflective in the UV-C range;
an inward facing surface of one or more of the lower wall, side wall and lid comprising with reflectance of 90% in the UV-C range;
an inward facing surface of one or more of the lower wall, side wall and lid comprising with diffuse reflectivity>87% in the UV-C range;
an inward facing surface of one or more of the lower wall, side wall and lid comprising with reflectance of 90% to UV of wavelength 250 nm;
an inward facing surface of one or more of the lower wall, side wall and lid comprising with diffuse reflectivity>87% to UV of wavelength 250 nm;
an inward facing surface of one or more of the lower wall, side wall and lid comprising a protective surface coating applied thereto;
an inward facing surface of one or more of the lower wall, side wall and lid comprising a protective polymer film applied thereto, for example a polyethylene film;
an inward facing surface of one or more of the lower wall, side wall and lid comprising a protective polymer film, for example a polyethylene film;
an inward facing surface of one or more of the lower wall, side wall and lid comprising a protective film with thickness in the range 30 μm to 80 μm, for example in the range 50 μm to 60 μm;
an inward facing surface of two or three of the lower wall, side wall and lid having the same optical properties as one another, for example comprising the same material and/or surface finish as one another;
a sensor assembly to detect lighting conditions in the sterilization volume when the light source is operational;
a sensor assembly to detect lighting conditions in the sterilization volume when the light source is operational, wherein the sensor assembly comprises a plurality of sensors;
a controller configured to selectively actuate a light source;
a controller is configured to compare the lighting conditions detected by the sensor assembly, and in response provide feedback control for a sterilization operation;
a display to indicate operational statuses of the light source as user feedback;
an actuator configured to provide user input to a controller to activate a light source.

According to another aspect of the present disclosure, a storage compartment for a vehicle includes a body defining a sterilization volume configured to receive an item to be sterilized. The body includes a lower wall, and a side wall extending from the lower wall. A lid is coupled with at least one of the walls and is configured to selectively close the sterilization volume. A lighting assembly is provided, comprising at least one light source to direct emitted light into the sterilization volume. The inward facing surface of one or more of the lower wall, side wall and lid comprises an optically matt finish, such as an optically matt finish in the visible spectral range.

Embodiments of this aspect of the disclosure can include any one or a combination of the following features:
the lighting assembly comprising a plurality of light sources;
the lighting assembly comprising a plurality of light sources in the lid;
the lighting assembly comprising a plurality of light sources in the lower wall;
the inward facing surface of one or more of the lower wall, side wall and lid comprises a reflective finish in the UV-C range;
an inward facing surface of one or more of the lower wall, side wall and lid comprising a metallic material, such as an aluminum material with an anodized coating and/or a physical vapor deposition finish applied thereto in order to provide an optically matt finish in the visible range, but a finish that is reflective in the UV-C range;
an inward facing surface of one or more of the lower wall, side wall and lid comprising with reflectance of 90% in the UV-C range;
an inward facing surface of one or more of the lower wall, side wall and lid comprising with diffuse reflectivity>87% in the UV-C range;
an inward facing surface of one or more of the lower wall, side wall and lid comprising with reflectance of 90% to UV of wavelength 250 nm;
an inward facing surface of one or more of the lower wall, side wall and lid comprising with diffuse reflectivity>87% to UV of wavelength 250 nm;
an inward facing surface of one or more of the lower wall, side wall and lid comprising a protective surface coating applied thereto;
an inward facing surface of one or more of the lower wall, side wall and lid comprising a protective polymer film applied thereto, for example a polyethylene film;
an inward facing surface of one or more of the lower wall, side wall and lid comprising a protective polymer film, for example a polyethylene film;
an inward facing surface of one or more of the lower wall, side wall and lid comprising a protective film with thickness in the range 30 μm to 80 μm, for example in the range 50 μm to 60 μm;
an inward facing surface of two or three of the lower wall, side wall and lid having the same optical properties as one another, for example comprising the same material and/or surface finish as one another.

According to an aspect of the present disclosure, a storage compartment for a vehicle includes a body defining a sterilization volume configured to receive an item to be sterilized. The body includes a lower wall, and a side wall extending from the lower wall. A lid is coupled with at least one of the walls and is configured to selectively close the sterilization volume. A lighting assembly is provided, comprising at least one light source to direct emitted light into the sterilization volume. A support is positioned within the sterilization volume. The support is operably coupled with the body and comprises a plurality of openings to allow light from the light source to pass therethrough.

Embodiments of this aspect of the disclosure can include any one or a combination of the following features:
the lighting assembly comprising a plurality of light sources;
the lighting assembly comprising a plurality of light sources in the lid;
the lighting assembly comprising a plurality of light sources in the lower wall;
the inward facing surface of one or more of the lower wall, side wall and lid comprising an optically matt finish in the visible spectral range;
the inward facing surface of one or more of the lower wall, side wall and lid comprises a reflective finish in the UV-C range;
the support removably coupled within the sterilization volume;
the support comprising a plurality of openings therethrough to allow light from the light sources to pass therethrough;
the openings having a characteristic dimension of between 5 mm and 10 mm, such as 8 mm, the characteristic dimension being for example the length of one or more sides of the openings;
the openings being generally rectangular when viewed in the plane of the support, for example generally square;
the support formed from a mesh material;
the support comprising a wire mesh;
the support comprising a mesh formed of 1 mm diameter wire;
the support comprising a mesh of metallic material;
the support comprising a stainless steel;
the support comprising a 304 grade stainless steel;
the support formed as a basket;
the support comprising an electro-chemically polished finish; —the support defining a support portion for the item to be sterilized, such as on which the item to be sterilized rests, in use;
the support portion being spaced from the lower wall and spaced from the lid when the sterilization volume is closed, such as substantially midway between lower wall and the lid.
the support portion being substantially parallel to the lower wall;
the support portion being spaced from the side wall;
the support portion being substantially central to the sterilization volume;
the support comprising a coupling portion extending from support portion, thereby the support is coupled to side wall;
the coupling portion extending from the support portion at an angle to the support portion, for example substantially perpendicular to the support portion;
the coupling portion extending from more than one side of the support portion;
the coupling portion extending to couple with the side wall, such as proximate to the lid
the coupling portion extending to couple to the side wall at the top of the side wall;
the support comprising an optically matt finish in the visible spectral range;
the support portion comprising an out of plane displacement, such as a displacement above, or a displacement below the support plane;
the out of plane displacement resulting in a non-planar perimeter for openings in the support portion;
the support portion comprising a plurality of out of plane displacements;
the support portion comprising a plurality of out of plane displacements;
the support portion comprising an out of plane displacement projecting toward the lower wall;
the support portion comprising an out of plane displacement projecting toward the lower wall;
the support portion comprising a curved surface, such as without discontinuity of curvature;
the support portion comprising a ripple pattern, with an out of plane displacement in the form of a ripple projecting towards the lower wall, and/or an out of plane displacement in the form of a ripple projecting toward the lid;
the support portion comprising a ripple pattern, with ripples projecting towards the lower wall, and/or ripples projecting toward the lid;
the support portion comprising ripple pattern, with a first arrangement of ripples projecting toward the lower wall and/or a second arrangement of ripples projecting toward the lid;
the support portion comprising a ripple pattern, with ripples of the first arrangement of ripples arranged between ripples of the second arrangement of ripples;
the support portion comprising a two-dimensional ripple pattern, with ripples of the first arrangement of ripples arranged between ripples of the second arrangement of ripples considering a first direction, and considering a second direction non-parallel to the first direction;
the support portion comprising a two-dimensional ripple pattern, with ripples of the first arrangement of ripples arranged between ripples of the second arrangement of ripples considering a first direction, and considering a second direction perpendicular to the first direction.

According to an aspect of the present disclosure, a storage compartment for a vehicle includes a body defining a sterilization volume configured to receive an item to be sterilized. The body includes a lower wall, and a side wall extending from the lower wall. A lid is coupled with at least one of the walls and is configured to selectively close the sterilization volume. A lighting assembly is provided, comprising at least one light source to direct emitted light into the sterilization volume. A sensor assembly is provided to detect lighting conditions in the sterilization volume when the light source is operational. A controller is configured to compare the lighting conditions detected by the sensor assembly, and in response control the sterilization operation.

Embodiments of this aspect of the disclosure can include any one or a combination of the following features:
the sensor assembly comprises a plurality of sensors;
the controller is arranged to provide feedback control for a sterilization operation;
the controller is arranged to control the vary the duration of the sterilization operation according to the lighting conditions detected by the sensor assembly;
the controller is arranged to output an expected total duration of a sterilization operation for display to a user, according to the lighting conditions detected by the sensor assembly;
the controller is arranged to output an expected remaining duration of a sterilization operation for display to a user, according to the lighting conditions detected by the sensor assembly;
the controller is arranged to output an indication of completion of a sterilization operation, on completion of a sterilization operation.

These and other aspects, objects, and features of the present disclosure will be understood and appreciated by those skilled in the art upon studying the following specification, any claims, and appended drawings.

### BRIEF DESCRIPTION OF THE DRAWINGS

The following is a description of the figures in the accompanying drawings. The figures are not necessarily to scale, and certain features and certain views of the figures may be shown exaggerated in scale or in schematic in the interest of clarity and conciseness.

In the drawings:

FIG. 1 is a side perspective view of a vehicle interior including a center stack portion and a center console, according to various examples;

FIG. 2A is a schematic view of a storage compartment for a vehicle according to various examples, viewed from front, one side and above;

FIG. 2B is a schematic view of a storage compartment for a vehicle according to various examples, viewed from front, one side and below;

FIGS. 3A, 3B and 3C are schematic front cross-sectional views of a storage compartment for a vehicle, from front, front one side and above and front one side and below respectively, according to various examples;

FIGS. 4A and 4B are schematic side cross-sectional views of a storage compartment for a vehicle, from one side rear and above, and one side rear and below respectively, according to various examples;

FIG. 5 is a schematic plan cross-sectional view of a storage compartment for a vehicle, according to various examples, from above, one side and front;

FIG. 6 is a schematic underneath plan cross-sectional view of a storage compartment for a vehicle according to various examples;

FIG. 7 shows a storage compartment for a vehicle according to various examples, viewed from front and above, with lid open and support removed for illustrative purposes;

FIG. 8 shows a storage compartment for a vehicle according to various examples, viewed from front and above, with lid open and support in place;

FIG. 9 shows a support from front and below; and

FIG. 10 is a block diagram of an operating system for a storage compartment of a vehicle, according to various examples.

### DESCRIPTION OF EXAMPLE EMBODIMENTS

The present illustrated embodiments reside primarily in combinations of method steps and apparatus components related to a charging and disinfecting/cleaning storage compartment. Accordingly, the apparatus components and method steps have been represented, where appropriate, by conventional symbols in the drawings, showing only those specific details that are pertinent to understanding the embodiments of the present disclosure so as not to obscure the disclosure with details that will be readily apparent to those of ordinary skill in the art having the benefit of the description herein. Further, like numerals in the description and drawings represent like elements.

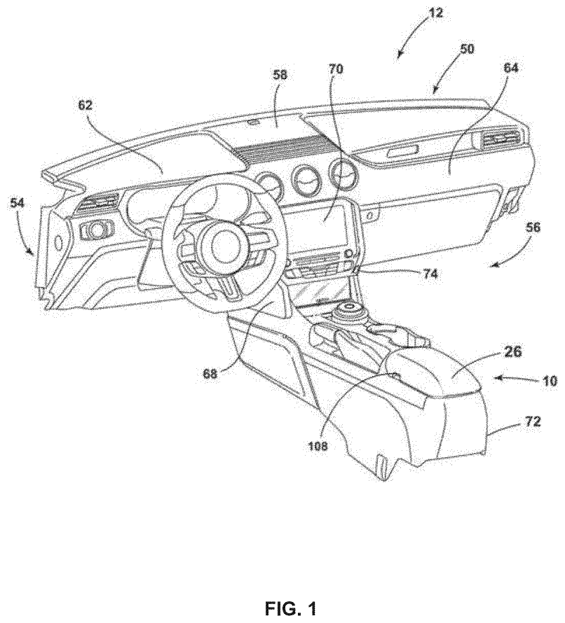

For purposes of description herein, the terms “upper”, “lower”, “right”, “left”, “rear”, “front”, “vertical”, “horizontal”, and derivatives thereof shall relate to the disclosure as oriented in FIG. 1. Unless stated otherwise, the term “front” shall refer to the surface of the element closer to an intended viewer, and the term “rear” shall refer to the surface of the element further from the intended viewer. However, it is to be understood that the disclosure may assume various alternative orientations, except where expressly specified to the contrary. It is also to be understood that the specific devices and processes illustrated in the attached drawings, and described in the following specification are simply exemplary embodiments of the inventive concepts defined in any appended claims. Hence, specific dimensions and other physical characteristics relating to the embodiments disclosed herein are not to be considered as limiting, unless the claims expressly state otherwise.

The terms “including”, “comprises”, “comprising”, or any other variation thereof, are intended to cover a non-exclusive inclusion, such that a process, method, article, or apparatus that comprises a list of elements does not include only those elements but may include other elements not expressly listed or inherent to such process, method, article, or apparatus. An element proceeded by “comprises a . . . ” does not, without more constraints, preclude the existence of additional identical elements in the process, method, article, or apparatus that comprises the element.

Referring to FIG. 1, reference numeral 10 generally designates a storage compartment for a vehicle 12. The storage compartment 10 includes a body defining a sterilization volume. The sterilization volume is configured to receive an item to be sterilized, such as a mobile phone.

FIG. 1 shows the storage compartment 10 in context within the vehicle 12. A vehicle interior 50 is generally illustrated including a driver's side 54 and a passenger's side 56. An instrument panel 58 spans laterally across the front of the vehicle interior 50. The instrument panel 58 may be divided into a driver's side portion 62, a passenger's side portion 64, and a center stack portion 68. Each of these portions 62, 64, 68 may serve different functions and include various electrical and mechanical components. A display 70 may be positioned on the center stack portion 68. A center console 72 may be coupled to the center stack portion 68. The center console 72 extends vehicle rearward from the center stack portion 68 and is positioned between the driver side 54 and the passenger side 56.

The center console 72 includes the storage compartment 10, the storage compartment having an opening at the top, to be selectively opened or closed by a lid 26. The storage compartment 10 may include at least one support positioned within the sterilization volume of the storage compartment 10, as set out in more detail below. In various examples, the storage compartment 10 may be positioned to be accessible through the opening at the top by an occupant positioned in either the driver side portion 62 or the passenger side portion 64 of the vehicle interior 50.

The storage compartment 10 may be configured to sterilize, including to disinfect and/or clean items (e.g., wallets, pens, keys, etc.) positioned within the storage compartment. The lid 26 is positioned to selectively close the storage compartment 10 to seal the storage compartment 10 when the storage compartment 10 is in use and/or to conceal the storage compartment 10 when the storage compartment 10 is not in use.

The lid 26 may be mounted on a horizontally positioned hinge. However, it is contemplated that the lid 26 may be coupled with any portion of the body by any type of hinge without departing from the scope of the present disclosure. When the lid 26 is open, the lid 26 may extend upwardly. When the lid 26 is closed as shown in FIG. 1, the lid 26 may be substantially parallel to the lower wall and may be configured to cover an open end of the storage compartment 10. The lid 26 may include an outer cover, such as shown in FIG. 1. The outer cover may be formed of a material similar to materials used for the center stack portion 68 and/or center console 72. The material selected for the outer cover may be selected to achieve a continuous aesthetic. Alternatively, the outer cover of the lid 26 may be formed of a different material than the material used for the center stack portion 68 and/or center console 72. A handle 108 may be positioned on the outer cover of the lid 26. The lid 26 may include any closing and/or latching mechanism configured to maintain the lid 26 in an open position or a closed position without departing from the scope of the present disclosure. When the lid 26 is closed, the lid 26 may be configured to seal the storage compartment 10 and prevent leaking of light from the storage compartment 10, as discussed elsewhere herein.

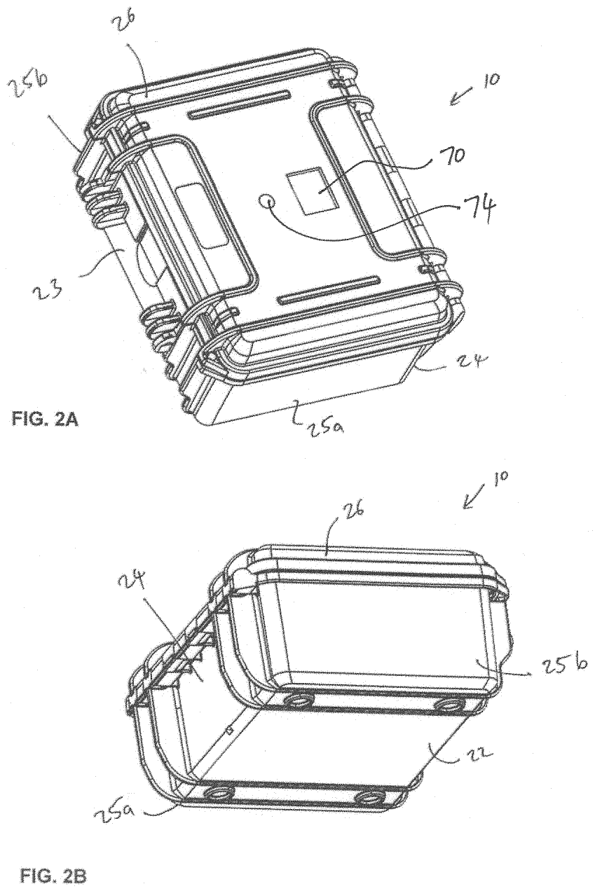

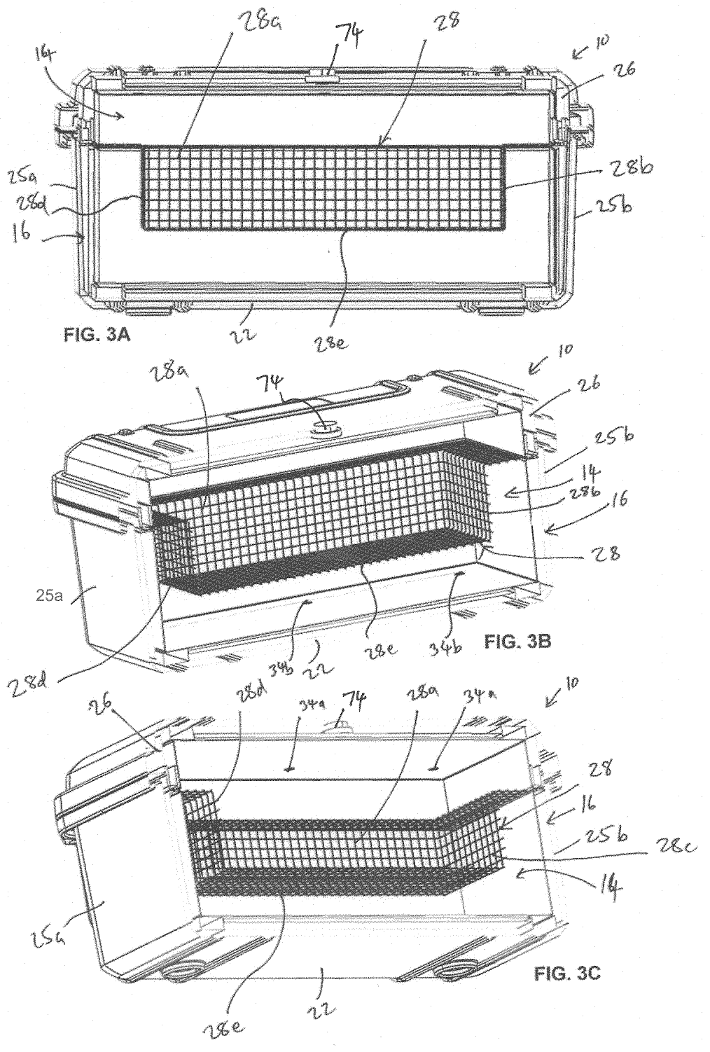

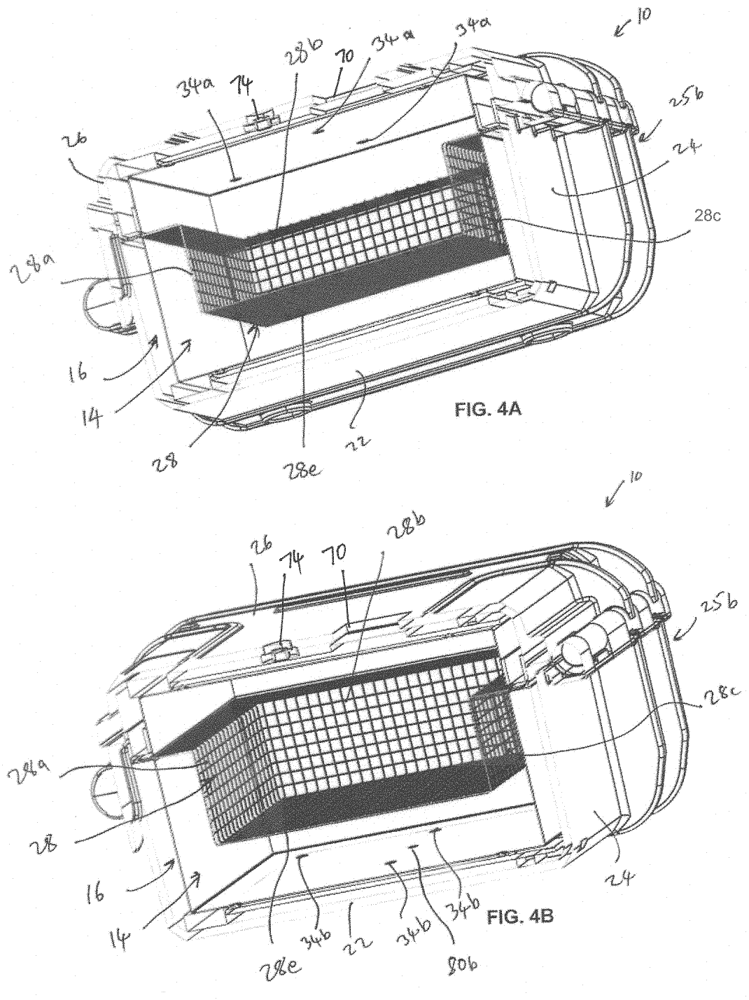

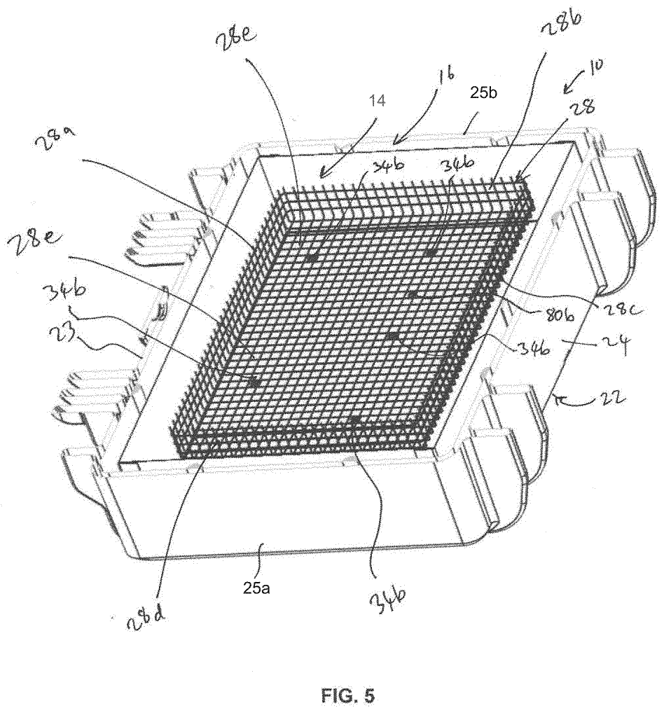

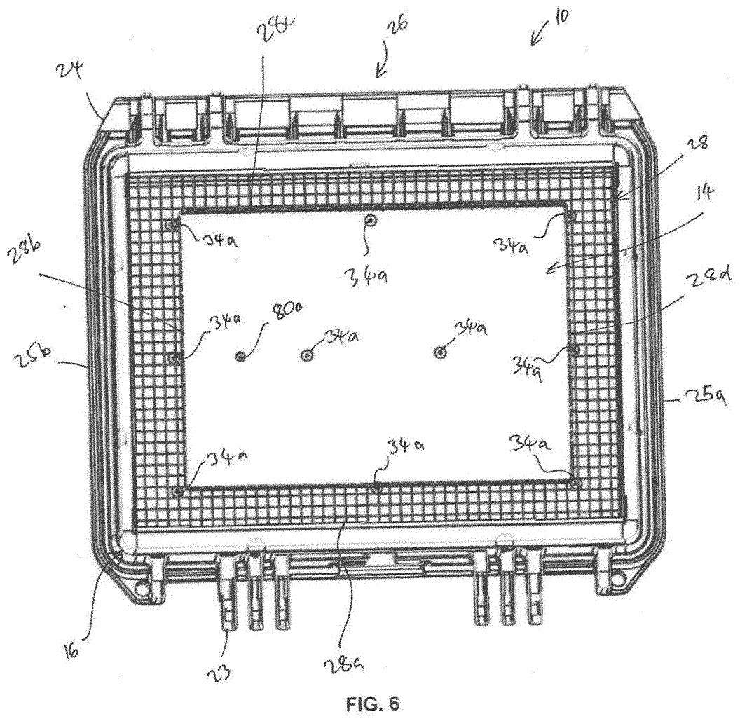

Within the center console 72, and under the outer cover, the arrangement of the lid 26, and its connection with the body defining the sterilization volume will be described with reference to FIG. 2 through FIG. 6.

Referring to FIG. 2 through FIG. 7, the body 16 includes a lid 26, a lower wall 22, a front wall 23, a rear wall 24 and first and second side walls 25a, 25b. These are inward facing, toward the center of the sterilization volume.

The lid 26 is hingedly coupled with the body 16 and is configured to selectively seal the sterilization volume 14. A support 28 is removably positioned within the sterilization volume 14. The support 28 comprises first through fourth side walls 28a-28d and a base 28e. The walls and base of the support 28 comprise a mesh material, and together form a basket in which items to be sterilized may be positioned. The base 28e forms a support portion of the support 28, and the first through fourth side walls 28a-28d form a coupling portion of the support 28.

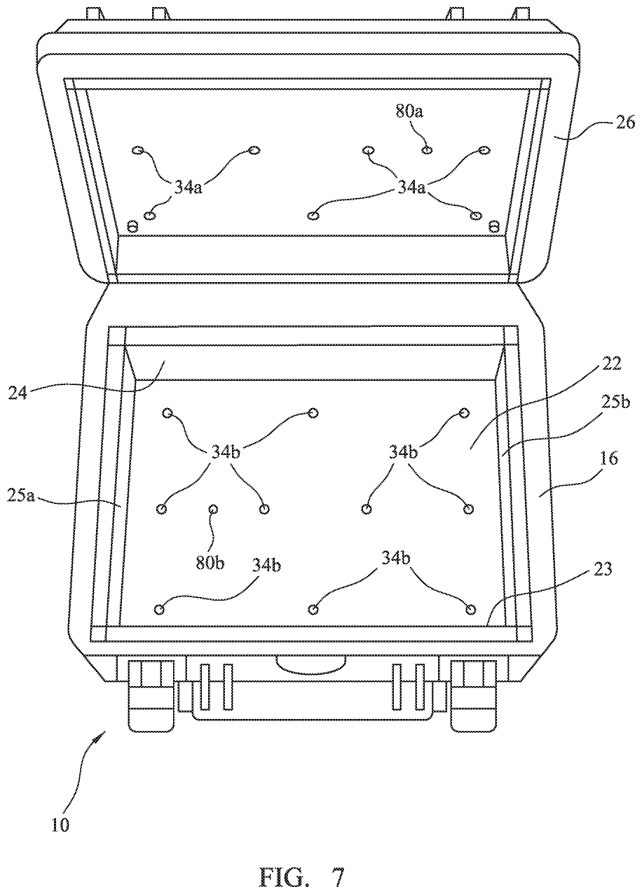

The support 28, which has been removed in FIG. 7, is configured to cooperate with the body 16 so as to be retained in the sterilization volume, with the walls and base of the support separated from the walls of the body 16.

An upper lighting assembly is positioned in the lid 26 of the body 16, and a lower lighting assembly is positioned in the lower wall 22 of the body 16. The upper and lower lighting assemblies each includes a plurality of light sources 34a, 34b respectively, positioned to direct emitted light into the sterilization volume 14.

An upper sensor 80a is positioned in the lid 26 of the body 16, and a lower sensing assembly 80b is positioned in the lower wall 22 of the body 16. The upper and lower sensing assemblies each include a sensor positioned to detect light emitted into the sterilization volume from the lighting assemblies.

In various examples, a support 28 may be positioned within the sterilization volume 14 of the storage compartment 10. The support 28 may be fixedly positioned within the sterilization volume 14 of the storage compartment 10 or may be removable from the sterilization volume 14 of the storage compartment 10. The support 28 may be formed of a mesh of metallic or polymeric material, or any other material/composite and may be configured to support and/or align items to be sterilized within the storage compartment 10.

The pluralities of light sources 34a, 34b are positioned to illuminate the sterilization volume 14 and may be operably coupled with the body 16. The plurality of light sources 34a, 34b is configured to direct and provide a substantially even distribution of the emitted light into the sterilization volume 14. The light sources 34a, 34b comprise LEDs that operate to emit in the ultraviolet, preferably UV-C range. It will be understood that any combination of light sources may be used. For example, the lighting assembly may include a first light sources or plurality of first light sources that are configured to emit a first emitted light having a first wavelength and second light source or plurality of light sources that are configured to emit a second emitted light having a second wavelength. For example, the first light source(s) may be an ultraviolet light emitting diode configured to produce emitted light having a wavelength within the UV light range with a first spectral intensity distribution. The emitted light within the UV light range is configured to disinfect an item or object such as a mobile phone, keys, etc. positioned within the storage compartment 10. It will be understood than any or all of the plurality of light sources may be configured to produce emitted light within the UV light range upon actuation.

The support 28 may be selectively positioned within the sterilization volume 14 of the storage compartment 10. The support 28 is provides at its base a support portion formed by the lower wall 28e on which an item to be sterilized may rest. Openings in the support 28 allow UV light to pass through the support, facilitating distribution of UV light in the sterilization volume and enabling more than one side of an item to be sterilized to be irradiated.

In some examples, the internal surfaces and the storage compartment 10 may be provided from materials that reflect ultraviolet radiation emitted by the light sources, to improve distribution of light energy within the sterilization volume and to prevent light from escaping from the storage compartment. The internal surfaces may also optionally include a protective layer to prevent physical damage and prevent degradation of the reflective characteristics of the materials within the storage compartment.

The internal surfaces and the storage compartment 10 comprise a chemically anodized and PVD coated aluminum sheet, of a type supplied by ALANOD GmbH & Co. KG, of Ennepetal, Germany. Suitable products are referred to as “UV-C High Specular PVD Coated aluminium sheet”, such as MIRO® 20 UVC product. This enables the internal surface of the storage compartment to be highly reflective to UV-C. A fine matt finish aids scattering, and make the material more resistant to marking or other damage handling etc.

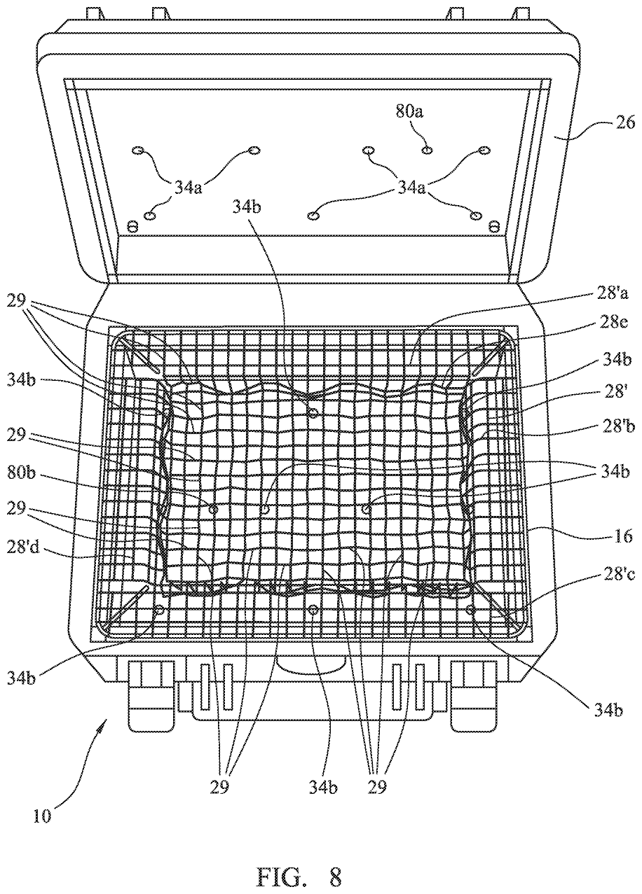

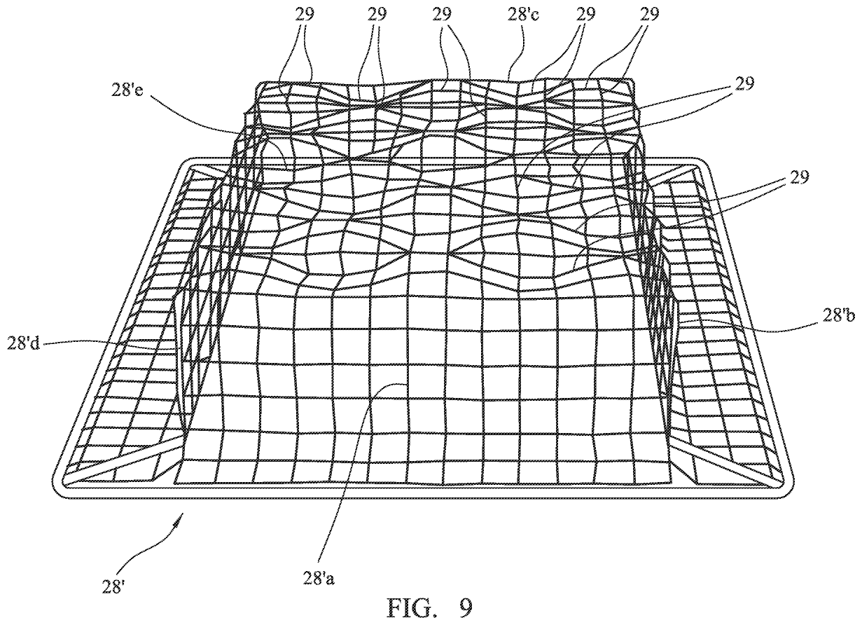

FIGS. 8 and 9 show an embodiment of support 28 that reduce shadowing of items therein considering the mesh material of the support 28′ itself and the positioning of base 28e′ on which the item to be sterilized rests between the light sources 34b and the item to be sterilized. The form provided for the support 28′ aids penetration of the UV-C into the sterilization volume, in particular to the part of the sterilization volume within the support 28′.

As shown in FIGS. 8 and 9 the base is generally planar, but with a plurality of out of plane displacements 29 arranged in a ripple pattern to extend towards the lower wall and the lid. For the sake of clarity, not all of the displacements 29 are labelled. As will be appreciated, the ripple pattern comprises an upper extent which defines the support plane on which an item to be sterilized rests. The displacements help maintain structural integrity of the support 28′ and enable both relatively large and relatively small items to be supported thereon, while reducing direct shading from the mesh material of the support 28′.

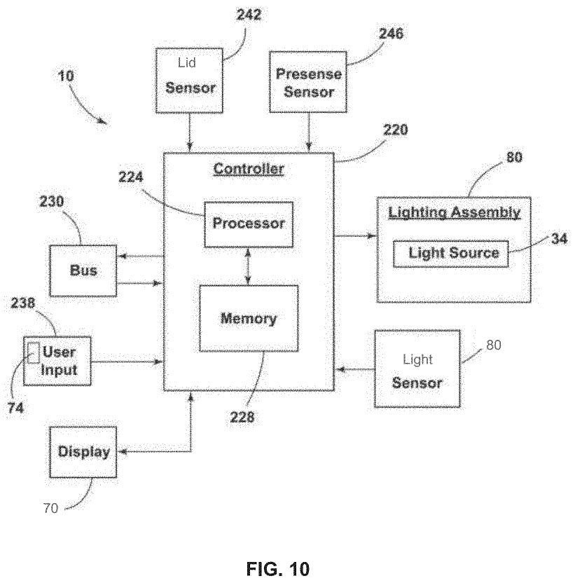

Referring now to FIG. 10, a block diagram of the operating system 40 of the storage compartment 10 is shown having control circuitry exemplarily illustrated as a controller 220 including a processor 224 and memory 228. The controller 220 may include other or additional analogue and/or digital circuitry. The controller 220 may process input information from the memory 228 and generate an output to the lighting assembly 30.

The controller 220 may be configured to receive various inputs from the vehicle 12 via a communication bus 230 and the lighting assembly 30. For example, the inputs may include a feedback signal from the lighting assembly 30 identifying a disinfecting status, a disinfecting and cleaning status, and/or light source functionality.

In some embodiments, the lighting assembly 30 may comprise a sensor 246 configured to sense a presence or absence of an item to be sterilized. The sensor 246 may be configured to detect a presence of the mobile phone or other item to be sterilized.

The operating system 40 of the storage compartment 10 may further comprise at least one user input 238 in communication with the controller 220. For example, the actuator 74 may provide a user input 238. The user input 238 may be configured to initiate a disinfecting function. In response to receiving a signal from the user input 238, the controller 220 may activate the light source. The light source emits emitted light within the UV light range which is configured to sterilize and sanitize an outer surface of items in the storage compartment. Further, the emitted light is configured to clean the outer surface of items in the storage compartment and the exposed inner surfaces of the storage compartment 10. The controller 220 may be configured to maintain the activation of the light source 34a to emit the emitted light 36a for a predetermined period of time that may correspond to a disinfection period. For example, the controller 220 may be configured to maintain the activation of the light source for a time frame for disinfecting. The time frame may be about 1 minute to about 5 minutes or any time increment, or range of time increments therebetween (e.g., 2 minutes). The emitted light may also be configured to identify and warn an occupant of the vehicle 12 that a disinfecting function is being processed in sterilization volume 14. When the controller 220 activates the light sources of the lighting assembly, the light source may be illuminated for predetermined time increments.

In some embodiments a light sensor 80 is provided in the sterilization volume. By measuring the amount of light within the sterilization volume, accounting for obstruction and absorption by items to be sterilized, it is possible to adjust the amount of time for which the light source needs to operate. If the light sensor 80 detects that the storage compartment is overloaded, such the effective sterilization may not be achieved even with a relatively long period of operation of the light source, then a warning signal can be raised by the controller 220, for example displayed on the display 70.

In some embodiments, the operating system 40 of the storage compartment 10 may further comprise a lid sensor 242 in communication with the controller 220. The lid sensor 242 may comprise a switch, for example, a magnetic or other proximity switch, located proximate a closure surface corresponding to the lid 26 and the body 16 of the storage compartment 10. The lid sensor 242 may be configured to output a signal to the controller 220 in response to the lid 26 being open or closed. In this way, the controller 220 may control the light sources such that the emitted light within the UV light range is only output when the lid 26 is closed.

The presence sensor 246 may be disposed in the body 16 of the storage compartment 10. For example, the presence sensor 246 may be positioned proximate the lower wall 22 of the body 16. The presence sensor 246 operates to identify the presence of a mobile phone and/or any other object that may be positioned within the sterilization volume 14. The presence sensor 246 may comprise a capacitive sensor, reflective sensor, light sensor, a weight sensor, or any other form of sensor operable to detect the presence of a mobile phone and/or any other object in the sterilization volume 14. In response to the mobile phone and/or another object being located within the sterilization volume 14, the presence sensor 246 may send a signal to the controller 220. In response to receiving the signal from the presence sensor 246, the controller 220 may activate the lighting assembly 30.

In various examples, the user input 238 may include the actuator 74 positioned on or proximate the center stack portion 68 and/or center console 72. The actuator 74 may be a switch, button, knob, or any other actuator configured to provide input to the controller 220. The actuator 74 may have various states including, for example, “Off”, “Sterilize” etc. The actuator 74 may be positioned proximate the display 70. The display 70 may be operably coupled with the controller 220 so that the controller 220 may actuate the display 70 to indicate the status of the storage compartment 10. The controller 220 may further actuate the display 70 to indicate remaining time duration for the sterilization cycle, or completion of the sterilization cycle when the remaining time duration reaches zero. In the embodiments of FIG. 2 to FIG. 8, the actuator 74 comprises a push button provided on the lid 26. In the embodiments of FIG. 2 to FIG. 8, the display 70 is provided on the lid 26.

The use of the lighting assembly 30 provides a storage compartment 10 that can disinfect and/or clean objects positioned within the sterilization volume 14 of the storage compartment 10. This allows a user to easily disinfect and/or clean objects using the emitted light of the UV light source(s).

It will be understood by one having ordinary skill in the art that construction of the described invention and other components is not limited to any specific material. Other exemplary examples of the invention disclosed herein may be formed from a wide variety of materials, unless described otherwise herein.

For purposes of this disclosure, the term “coupled” (in all of its forms, couple, coupling, coupled, etc.) generally means the joining of two components (electrical or mechanical) directly or indirectly to one another. Such joining may be stationary in nature or movable in nature. Such joining may be achieved with the two components (electrical or mechanical) and any additional intermediate members being integrally formed as a single unitary body with one another or with the two components. Such joining may be permanent in nature or may be removable or releasable in nature unless otherwise stated.

As used herein, the term “about” means that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art. When the term “about” is used in describing a value or an end-point of a range, the disclosure should be understood to include the specific value or end-point referred to. Whether or not a numerical value or end-point of a range in the specification recites “about”, the numerical value or end-point of a range is intended to include two embodiments: one modified by “about”, and one not modified by “about”. It will be further understood that the end-points of each of the ranges are significant both in relation to the other end-point, and independently of the other end-point.

The terms “substantial”, “substantially”, and variations thereof as used herein are intended to note that a described feature is equal or approximately equal to a value or description. For example, a “substantially planar” surface is intended to denote a surface that is planar or approximately planar. Moreover, “substantially” is intended to denote that two values are equal or approximately equal. In some embodiments, “substantially” may denote values within about 10% of each other.

It is also important to note that the construction and arrangement of the elements of the disclosure as shown in the exemplary embodiments is illustrative only. Although only a few embodiments of the present innovations have been described in detail in this disclosure, those skilled in the art who review this disclosure will readily appreciate that many modifications are possible (e.g., variations in sizes, dimensions, structures, shapes and proportions of the various elements, values of parameters, mounting arrangements, use of materials, colors, orientations, etc.) without materially departing from the novel teachings and advantages of the subject matter recited. For example, elements shown as integrally formed may be constructed of multiple parts or elements shown as multiple parts may be integrally formed, the operation of the interfaces may be reversed or otherwise varied, the length or width of the structures and/or members or connector or other elements of the system may be varied, the nature or number of adjustment positions provided between the elements may be varied. It should be noted that the elements and/or assemblies of the system may be constructed from any of a wide variety of materials that provide sufficient strength or durability, in any of a wide variety of colors, textures, and combinations. Accordingly, all such modifications are intended to be included within the scope of the present innovations. Other substitutions, modifications, changes, and omissions may be made in the design, operating conditions, and arrangement of the desired and other exemplary embodiments without departing from the spirit of the present innovations.

It will be understood that any described processes or steps within described processes may be combined with other disclosed processes or steps to form structures within the scope of the present disclosure. The exemplary structures and processes disclosed herein are for illustrative purposes and are not to be construed as limiting.

What is claimed is:

1. A storage compartment for a vehicle, comprising:
a body defining a sterilization volume configured to receive an item to be sterilized, the body comprising a lower wall, and a side wall extending from the lower wall;
a lid coupled with the at least one of the walls, to selectively close the sterilization volume; and
a lighting assembly comprising a plurality of light sources in the lid and a plurality of light sources in the lower wall, the light sources configured to direct emitted light into the sterilization volume.

2. The storage compartment of claim 1, wherein the light source in the lid and the light sources in the lower wall comprise UV-C emitters.

3. The storage compartment of claim 1, wherein the light sources in the lid and the light sources in the lower wall are arranged in a spaced array.

4. The storage compartment of claim 1, comprising a removable support coupled within the sterilization volume.

5. The storage compartment of claim 4, wherein the support comprises a basket of mesh material.

6. The storage compartment of claim 4, wherein the support comprises a support portion being spaced from the lower wall and spaced from the lid when the sterilization volume is closed.

7. The storage compartment of claim 6, wherein the support portion comprises an out of plane displacement.

8. The storage compartment of claim 6, wherein the support portion comprises a ripple pattern, with out of plane displacements in the form of ripples projecting towards the lower wall.

9. The storage compartment of claim 6, wherein the support portion comprises a ripple pattern, with ripples of a first arrangement of ripples arranged between ripples of a second arrangement of ripples.

10. The storage compartment of claim 9, wherein the with ripples of the first arrangement of ripples arranged between ripples of the second arrangement of ripples considering a first direction, and considering a second direction that is not aligned parallel to the first direction.

11. The storage compartment of claim 4, wherein at least one light source is located directly above the support and at least one light source located directly below the support.

12. The storage compartment of claim 1, wherein an inward facing surface of one or more of the lower wall, side wall and lid comprising an optically matt finish in the visible spectral range.

13. The storage compartment of claim 1, comprising a sensor assembly to detect lighting conditions in the sterilization volume when the light source is operational.

14. The storage compartment of claim 13, wherein the sensor assembly comprises a plurality of sensors.

15. The storage compartment of claim 1, comprising a controller to selectively actuate a light source, to compare the lighting conditions detected by the sensor assembly, and in response provide feedback control for a sterilization operation.

16. A storage compartment for a vehicle, comprising:
a body defining a sterilization volume configured to receive an item to be sterilized, the body comprising a lower wall, and a side wall extending from the lower wall;
a lid coupled with at least one of the walls, to selectively close the sterilization volume; and
a lighting assembly comprising at least one light source to direct emitted light into the sterilization volume.

17. The storage compartment of claim 16, wherein the inward facing surface of at least one of the lower wall, side wall and lid comprises an optically matt finish in the visible range, but a reflective finish in the UV-C range.

18. The storage compartment of claim 16, comprising a support positioned within the sterilization volume, operably coupled with the body and comprising a plurality of openings to allow light from the light source to pass therethrough.

19. The storage compartment of claim 18, wherein the support comprises an electro-chemically polished finish.

20. A storage compartment for a vehicle comprising:
a body defining a sterilization volume configured to receive an item to be sterilized, the body comprising a lower wall, and a side wall extending from the lower wall;
a lid coupled with at least one of the walls, to selectively close the sterilization volume;
a lighting assembly comprising at least one light source to direct emitted light into the sterilization volume;
a sensor assembly to detect lighting conditions in the sterilization volume when the light source is operational; and
a controller to compare the lighting conditions detected by the sensor assembly, and in response control the sterilization operation.
